(19) United States
(12) Patent Application Publication
(10) Pub. No.: US 2025/0000383 A1
(43) Pub. Date: Jan. 2, 2025

(54) SIGNAL PROCESSING APPARATUS AND METHOD

(71) Applicant: SONY GROUP CORPORATION, TOKYO (JP)

(72) Inventors: KIYOSHI YOSHIKAWA, TOKYO (JP); YASUHIDE HYODO, TOKYO (JP)

(21) Appl. No.: 18/709,610

(22) PCT Filed: Nov. 4, 2022
(86) PCT No.: PCT/JP2022/041123

§ 371 Date: May 13, 2024

(30) Foreign Application Priority Data
Nov. 19, 2021 (JP) 2021-188909

(51) Int. Cl. A61B 5/0533

(52) U.S. Cl. CPC A61B 5/0533

(57) ABSTRACT

The present technology relates to a signal processing apparatus and a method capable of improving the integration accuracy of estimation of a user state using a multimodal sensor. The signal processing apparatus estimates signal qualities for respective modals each representing a type of a biological signal of a user, detects at least one of the modals having variation heterogeneity representing that variation of the biological signal is different from a plurality of the modals, estimates a sensitivity to biological reaction of the modal based on a detection result of the modal having the variation heterogeneity, and integrally estimates a state of the user on the basis of the signal qualities and the sensitivities to the biological reaction. The present technology can be applied to a user state estimation processing system.

### TECHNICAL FIELD

The present technology relates to a signal processing apparatus and a method, and more particularly, to a signal processing apparatus and a method capable of improving the integration accuracy of estimation of a user state using a multimodal biological sensor.

### BACKGROUND ART

In a system for estimating a user state, in order to apply the system to general-purpose applications and improve resistance to body motion noise, it is desirable to provide a multimodal biological sensor so as to integrally perform processing of signals regarding emotions of a large number of living bodies (hereinafter, referred to as biological signals) acquired from the biological sensor.

Note that, a modal represents a type of the biological signal, for example, an electroencephalogram (EEG), an optical measurement of a volume change of a blood vessel (photoplethysmography (PPG)), variation in skin conductivity (electrodermal activity (EDA)) or the like. A biological sensor that senses such a plurality of types of biological signals is referred to as a multimodal biological sensor.

However, in the multimodal biological sensor, there is no guarantee that data of good signal quality can be acquired at all times, and furthermore, there is an individual difference (difference in a sensitivity to physiological reaction, for example, no-responder in perspiration) for each modal.

In the technology described in Patent Document 1, various feature amounts calculated for data acquired from a multimodal biological signal measurement device are used without considering individual differences, individual characteristics, and the like.

### CITATION LIST

### Patent Document

Patent Document 1: Japanese Patent Application Laid-Open No. 2009-142635

### SUMMARY OF THE INVENTION

### Problems to be Solved by the Invention

As described above, in a system using a multimodal biological sensor, it is required to estimate a user state in consideration of individual differences and individual characteristics.

The present technology has been made in view of such a situation, and an object thereof is to improve the integration accuracy of estimation of a user state using the multimodal biological sensor.

### Solutions to Problems

A signal processing apparatus according to one aspect of the present technology includes a signal processing unit that estimates signal qualities for respective modals each representing a type of a biological signal of a user, a sensitivity estimation unit that detects at least one of the modals having variation heterogeneity representing that variation of the biological signal is different from a plurality of the modals, and estimates a sensitivity to biological reaction of the modal based on a detection result of the modal having the variation heterogeneity, and an integration estimation unit that integrally estimates a state of the user on the basis of the signal qualities and the sensitivities to the biological reaction.

In one aspect of the present technology, signal qualities are estimated for respective modals each representing a type of a biological signal of a user, at least one of the modals having variation heterogeneity representing that variation of the biological signal is different from a plurality of the modals is detected, a sensitivity to biological reaction of the modal based on a detection result of the modal having the variation heterogeneity is estimated, and a state of the user is integrally estimated on the basis of the signal qualities and the sensitivities to the biological reaction.

### BRIEF DESCRIPTION OF DRAWINGS

FIG. 1 is a diagram illustrating a configuration example of a user state estimation system according to an embodiment of the present technology.

FIG. 2 is a block diagram illustrating a first configuration example of a user state estimation unit.

FIG. 3 is a block diagram illustrating a configuration example of a sensor signal processing unit.

FIG. 4 is a flowchart illustrating processing of the user state estimation unit of FIG. 2.

FIG. 5 is a flowchart illustrating biological reaction sensitivity estimation processing in step S14 in FIG. 4.

FIG. 6 is a diagram illustrating a specific example of baseline section detection.

FIG. 7 is a diagram illustrating an example of a correction coefficient based on a type of application and physiological knowledge.

FIG. 8 is a block diagram illustrating a second configuration example of the user state estimation unit.

FIG. 9 is a flowchart illustrating processing of the user state estimation unit of FIG. 8.

FIG. 10 is a flowchart illustrating the biological reaction sensitivity estimation processing in step S54 in FIG. 9.

FIG. 11 is a diagram illustrating a learning efficiency improvement support system to which the present technology is applied.

FIG. 12 is a diagram illustrating each scene in a use case.

FIG. 13 is a block diagram illustrating a configuration example of a computer.

### MODE FOR CARRYING OUT THE INVENTION

Hereinafter, embodiments for embodying the present technology will be described. The description will be made in the following order.
1. System Configuration
2. First Embodiment (Late Fusion Type)
3. Second Embodiment (Early Fusion Type)
4. Use Case
5. Others

### 1. System Configuration

### <Configuration Example of User State Estimation System>

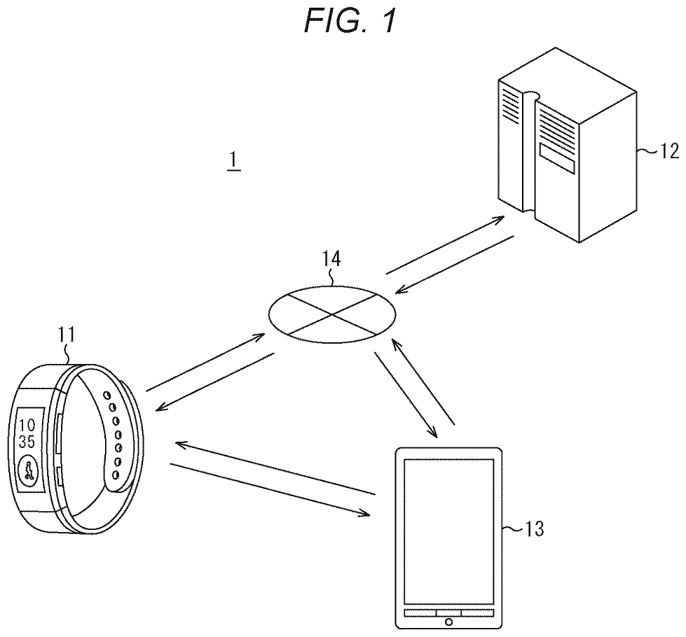

FIG. 1 is a diagram illustrating a configuration example of a user state estimation system according to an embodiment of the present technology.

A user state estimation system 1 of FIG. 1 includes a biological information processing device 11.

Note that, the user state estimation system 1 may include a server 12, a terminal device 13, and a network 14. In this case, in the user state estimation system 1, the biological information processing device 11, the server 12, and the terminal device 13 are connected to each other via the network 14.

The user state estimation system 1 is a system that detects biological signals and estimates a state (emotion) of the living body on the basis of the detected biological signals. For example, at least the biological information processing device 11 of the user state estimation system 1 is directly worn on the living body to detect biological signals.

The biological information processing device 11 is a wristband type device, for example, a wristwatch type, or the like and is worn on the wrist of a user.

The biological information processing device 11 includes one or a plurality of multimodal biological sensors that detect a plurality of types of biological signals including a perspiration state, a pulse wave, a myoelectric potential, a blood pressure, a blood flow, a body temperature, and the like of the user.

The biological information processing device 11 estimates a state of the user on the basis of the biological signals detected by the multimodal biological sensor. Based on the estimated state of the user, a concentration state, an awakening state, and the like of the user can be confirmed.

Note that, FIG. 1 illustrates the biological information processing device 11 that is a wristband type device to be worn on an arm, but the biological information processing device 11 is not limited to the example of FIG. 1.

For example, the biological information processing device 11 may be realized by a mode that can be worn on a part of a hand such as a wristband, a glove, a smart watch, or a ring. Furthermore, in a case where the biological information processing device 11 is in contact with a part of the living body such as a hand, the biological information processing device 11 may be formed to be included in an object that can be in contact with the user, for example. For example, the biological information processing device 11 may be provided on the surface of or inside an object that can be in contact with the user, such as a mobile terminal, a smartphone, a tablet, a mouse, a keyboard, a handle, a lever, a camera, an exercise tool (golf club, tennis racket, bow of archery, etc.), or a writing tool.

Furthermore, for example, the biological information processing device 11 may be realized in a form that can be worn on a part of the head or the ear of the user, such as a headband, a head-mounted display, headphones, earphones, a hat, an accessory, goggles, or glasses.

Note that, the wearing position and the wearing method of the biological information processing device 11 are not particularly limited as long as the biological information processing device 11 can detect a signal related to the state of the living body. For example, the biological information processing device 11 may not necessarily be in direct contact with the body surface of the living body. For example, the biological information processing device 11 may be in contact with the surface of the living body via clothing, a detection sensor protective film, or the like.

Furthermore, in the user state estimation system 1, the above-described biological information processing device 11 does not necessarily perform information processing by itself. For example, the biological information processing device 11 may include a biological sensor in contact with a living body, transmit biological signals detected by the biological sensor to other devices such as the server 12 or the terminal device 13, and the other devices may perform information processing on the basis of the received biological signals to estimate the state of the living body.

For example, in a case where the biological sensor is worn on the arm, head, or the like of the user, the biological information processing device 11 may transmit biological signals acquired from the biological sensor to the server 12 or the terminal device 13 including a smartphone or the like, and the server 12 or the terminal device 13 may perform information processing to estimate the state of the living body.

The biological sensor included in the biological information processing device 11 is in contact with the surface of the living body in various forms as described above to detect multimodal biological signals. Therefore, the measurement result of the biological sensor is easily affected by variation of the contact pressure between the biological sensor and the living body due to the body motion of the living body. For example, biological signals acquired from the biological sensor include noise caused by the body motion of the living body. It is desired to accurately estimate the state of the living body from the biological signals including such noise.

The body motion of the living body refers to the overall motion form when the living body moves, and examples thereof include motions of the living body such as twisting the wrist or bending and stretching fingers while the user wears the biological information processing device 11 on the wrist. Such motions of the user may vary the contact pressure between the biological sensor included in the biological information processing device 11 and the user.

Note that, in order to improve the accuracy of the biological signals acquired by the biological sensor, the biological information processing device 11 may include a second sensor or a third sensor to be described later in addition to the above-described biological sensor.

For example, the second sensor is configured to detect a body motion change of the living body. The third sensor is configured to detect a pressure change of the living body in the detection region of the biological sensor.

In this case, using a body motion signal and a pressure signal detected by the second sensor and the third sensor, the biological information processing device 11 can accurately reduce the body motion noise from the biological signals detected by the biological sensor. In the biological information processing device 11, using biological signals corrected in this manner, the user state estimation processing of the present technology to be described later may be performed.

In the user state estimation system 1, the server 12 includes a computer and the like. The terminal device 13 includes a smartphone, a mobile terminal, a personal computer, and the like.

The server 12 and the terminal device 13 receive information and signals transmitted from the biological information processing device 11 and transmit information and signals to the biological information processing device 11, via the network 14.

For example, as described above, the server 12 and the terminal device 13 receive the biological signals acquired by the biological sensor included in the biological information processing device 11 from the biological information processing device 11, and perform signal processing on the received biological signals to estimate the state of the living body.

The network 14 includes the Internet, a wireless local area network (LAN), and the like.

### 2. First Embodiment (Late Fusion Type)

### <First Configuration Example of User State Estimation Unit>

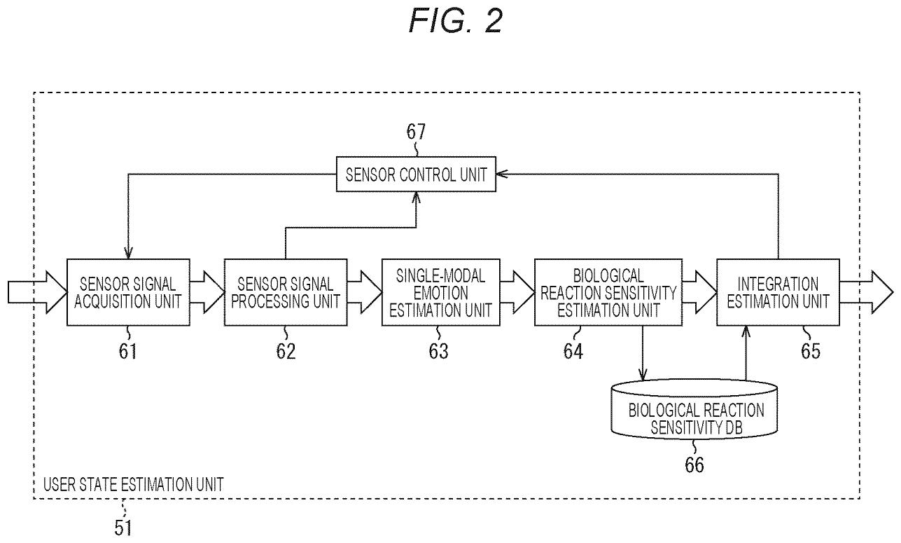

FIG. 2 is a block diagram illustrating a first configuration example of a user state estimation unit 51.

The user state estimation unit 51 of FIG. 2 is configured as a late fusion type in which user state estimation result for each modal is calculated and then the user state estimation results are integrated and output the final user state estimation result. As described above, the user state estimation unit 51 may be included in the biological information processing device 11, or may be included in the server 12 or the terminal device 13.

In FIG. 2, the user state estimation unit 51 includes a sensor signal acquisition unit 61, a sensor signal processing unit 62, a single-modal emotion estimation unit 63, a biological reaction sensitivity estimation unit 64, a biological reaction sensitivity database (DB) 65, an integration estimation unit 66, and a sensor control unit 67.

The sensor signal acquisition unit 61 acquires multimodal biological signals from each biological sensor of multimodal, and information associated with the living body (for example, acceleration information, gyro information, and the like of the wearing part) from the second sensor or the third sensor. The acquired biological signals and information associated with the living body are output to the sensor signal processing unit 62.

Furthermore, the sensor signal acquisition unit 61 turns off sensing of a modal with poor signal quality or turns off sensing of a modal with a poor sensitivity to biological reaction to be described later under the control of the sensor control unit 67. As a result, in a system requiring power saving such as a wearable environment, power saving can be realized without affecting the estimation accuracy of the user state.

The sensor signal processing unit 62 receives the biological signals of each modal from the sensor signal acquisition unit 61, performs preprocessing and signal quality estimation on the biological signals of each modal, and outputs, to the single-modal emotion estimation unit 63, a set of the preprocessed signals and the signal qualities that can be used in the subsequent processing.

### <Configuration of Sensor Signal Processing Unit>

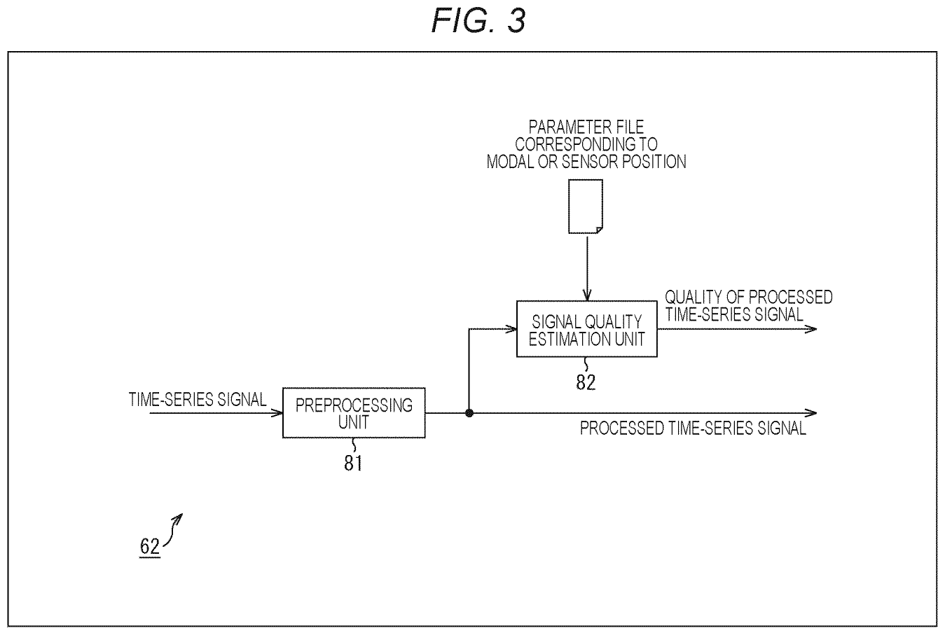

FIG. 3 is a block diagram illustrating a configuration example of the sensor signal processing unit 62.

The sensor signal processing unit 62 includes a preprocessing unit 81 and a signal quality estimation unit 82.

The preprocessing unit 81 performs preprocessing such as filtering, resampling, and denoising as necessary on a time-series signal acquired by a certain biological sensor and supplied from the sensor signal acquisition unit 61. The preprocessing unit 81 outputs the preprocessed time-series signal to the signal quality estimation unit 82 and the single-modal emotion estimation unit 63.

The signal quality estimation unit 82 estimates the quality of the preprocessed time-series signal supplied from the preprocessing unit 81, and outputs information indicating the estimated signal quality to the single-modal emotion estimation unit 63. The signal quality is represented by, for example, a numerical value from 0 to 1, where 0 indicates the worst quality and 1 indicates the best quality.

The signal quality estimation unit 82 can read a parameter file corresponding to the modal or the sensor position by using a general-purpose architecture such as a deep neural network (DNN). Using the read parameter file, the signal quality estimation unit 82 estimates the quality of the preprocessed time-series signal. As a result, the signal quality can be estimated regardless of the modal or the sensor position.

Information regarding the signal quality of the modal in which the signal quality estimated by the signal quality estimation unit 82 is worse than a certain threshold is notified to a sensor control unit 89 and sensing of the modal in which the signal quality is poor is temporarily turned off. As a result, power saving can be realized.

Returning to FIG. 2, the single-modal emotion estimation unit 83 receives the preprocessed time-series signal and the information indicating the signal quality supplied from the preprocessing unit 81 and the signal quality estimation unit 82, and performs user state estimation for a single modal using an estimation model corresponding to the modal.

The single-modal emotion estimation unit 83 outputs information indicating the quality of the input signal used for estimation to a subsequent biological reaction sensitivity estimation unit 64 together with the user state estimation result.

On the basis of the user state estimation result, the biological reaction sensitivity estimation unit 64 detects baseline sections of all modals. The baseline section means a section where the user state has been stable immediately before the user state to be estimated, and does not necessarily match with a section of the resting state in which the user is resting.

The biological reaction sensitivity estimation unit 64 detects a modal having variation heterogeneity from modals on the basis of variation properties and variation degree from the state in the baseline section in consideration of a reaction time constant for each modal. The variation heterogeneity indicates that at least one of properties or degree of variation is different from a plurality of modals. The reaction time constant is a constant representing the time required for reaction. For example, since the time required for the reaction varies depending on the modal, such as the reaction due to perspiration is fast while the reaction due to heartbeat is slow, in the biological reaction sensitivity estimation unit 64, the reaction time constant for each modal is considered.

The biological reaction sensitivity estimation unit 64 makes the properties of variation from the states in the baseline sections such as the variation direction from an uncomfortable state toward a comfortable state, and then detects a modal having variation heterogeneity from modals on the basis of variation amounts indicating how much the variation is.

For example, in a case where the recovery state of the user after the exercise is stopped is to be estimated, a just preceding section in which the user continues to exercise stably for a fixed period corresponds to the baseline section. After the exercise is stopped, on the basis of the state in the baseline section, variation heterogeneity of the estimation result of the recovery state of the user is detected for each modal and a modal having the variation heterogeneity is detected.

On the basis of the detection result of the variation heterogeneity of each modal (that is, the presence or absence of variation heterogeneity), the biological reaction sensitivity estimation unit 64 estimates the sensitivity to the biological reaction of each modal and registers, in the biological reaction sensitivity DB 65, the information indicating the sensitivity to the biological reaction of each modal.

The biological reaction sensitivity DB 65 stores the information indicating the sensitivity to the biological reaction estimated by the biological reaction sensitivity estimation unit 64, and the like. The information stored in the biological reaction sensitivity DB 65 is referenced by the integration estimation unit 66.

Note that, the biological reaction sensitivity DB 65 stores not only the information on a modal having variation heterogeneity but also the information on a modal having no variation heterogeneity.

As a result, the occurrence frequency of the variation heterogeneity can be calculated. Specifically, it is possible to distinguish whether or not the modal always has variation heterogeneity from the variation of other modals like a no-responder in perspiration, or whether or not the modal has variation heterogeneity occasionally depending on the contact condition of the perspiration sensor.

The integration estimation unit 66 integrates the single-modal emotion estimation result supplied from the single-modal emotion estimation unit 63 and the sensitivity to the biological reaction of each modal indicated by the information supplied from the biological reaction sensitivity estimation unit 64 or stored in the biological reaction sensitivity DB 65, to integrally estimate the user state. The integration estimation unit 66 outputs the user state estimation result to a control unit or a display control unit subsequent thereto (not illustrated).

In addition to the user state estimation result of each modal, the integration estimation unit 66 dynamically calculates the reliability based on the signal quality and the biological reaction sensitivity, and uses the calculated reliability as an index at the time of integration. That is, the integration estimation unit 66 integrates the states of the user for the respective modals with weights on the basis of the reliabilities of the states of the user for the respective modals using the signal qualities and the sensitivities to the biological reaction as indexes to integrally estimate the state of the user.

The sensitivity to the biological reaction of each modal is calculated on the basis of the occurrence frequency of the variation heterogeneity using the information indicating the latest sensitivity in the biological reaction sensitivity DB 65 as a base.

In the integration, the integration estimation unit 66 notifies the sensor control unit 67 of information regarding a modal having reliability sufficiently lower than a threshold, so that sensing of the modal is temporarily turned off. As a result, power saving can be realized.

The sensor control unit 67 receives information regarding a modal that does not contribute to user state estimation in the subsequent stages and the integration, supplied from the sensor signal processing unit 62 and the integration estimation unit 66, and notifies the sensor signal acquisition unit 61 of sensing off.

The sensor control unit 67 can check whether or not the signal quality has been improved due to change in a contact state, for example, by turning on the sensing of the modal turned off during a certain constant time period.

### <Processing of User State Estimation Unit>

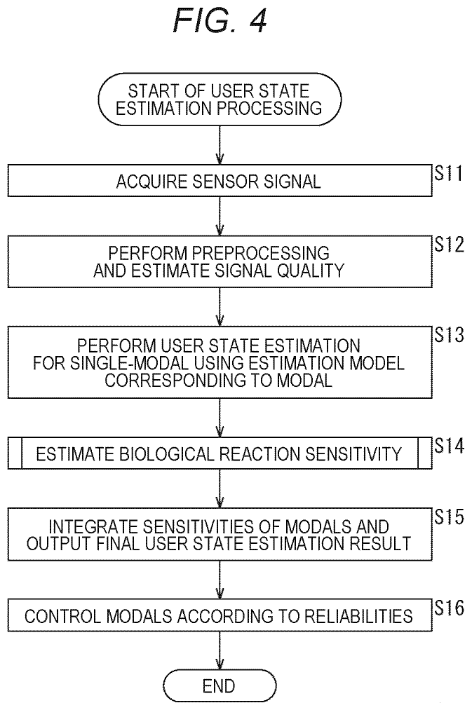

FIG. 4 is a flowchart illustrating user state estimation processing of the user state estimation unit 51 of FIG. 2.

In step S11, the sensor signal acquisition unit 61 acquires multimodal biological signals from the respective biological sensors of multimodal, and information associated with the living body from a second sensor or a third sensor.

In step S12, the sensor signal processing unit 62 receives the biological signals of the modals from the sensor signal acquisition unit 61, and performs preprocessing and signal quality estimation on the biological signals of the modals. The sensor signal processing unit 62 outputs, to the single-modal emotion estimation unit 63, a set of the preprocessed signals and the information indicating the signal quality, available in the subsequent processing.

In step S13, the single-modal emotion estimation unit 83 receives the preprocessed time-series signal and the information indicating the signal quality supplied from the signal quality estimation unit 82, and performs user state estimation for a single-modal using an estimation model corresponding to each modal.

The single-modal emotion estimation unit 83 outputs, together with the user state estimation result, information indicating the quality of the input signal used for estimation to the biological reaction sensitivity estimation unit 64.

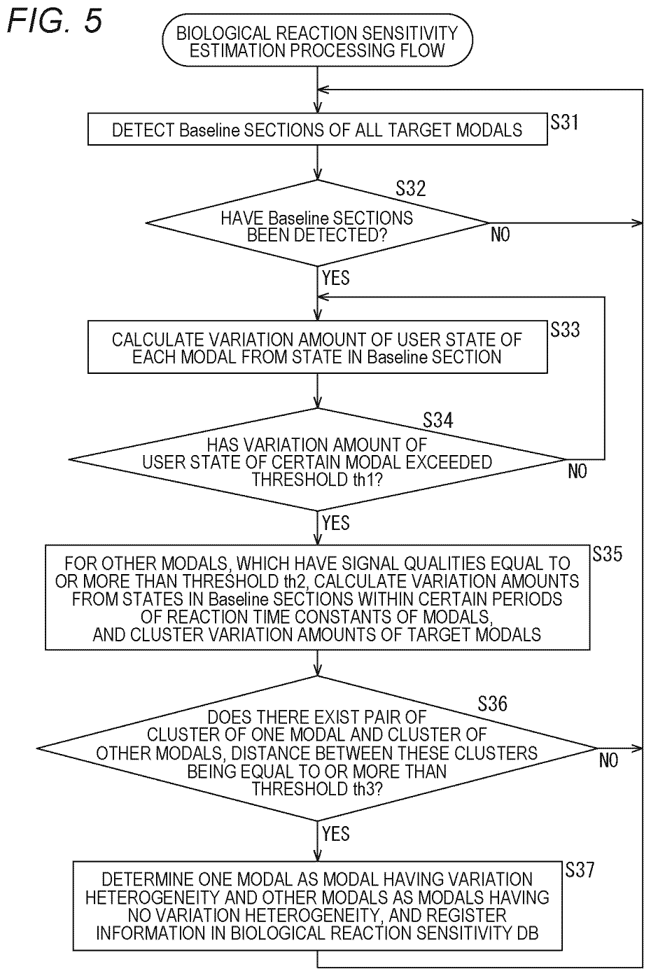

In step S14, the biological reaction sensitivity estimation unit 64 performs biological reaction sensitivity estimation processing on the basis of the user state estimation result. Details of the biological reaction sensitivity estimation processing will be described later with reference to FIG. 5. By the processing of step S14, the baseline sections of all modals are detected on the basis of the user state estimation result, a modal having variation heterogeneity from a plurality of the modals is detected on the basis of the variation properties and the variation degrees from the states in the baseline sections, and the information indicating the sensitivity to the biological reaction of each modal based on the presence or absence of the variation heterogeneity is registered in the biological reaction sensitivity DB 65.

In step S15, the integration estimation unit 66 integrates the single-modal state estimation results supplied from the single-modal emotion estimation unit 63 and the sensitivities to the biological reaction of the respective modals indicated by the information stored in the biological reaction sensitivity DB 65, and outputs the final user state estimation result. At this time, for the integration, the reliabilities of the states of the user for the respective modals using the signal qualities and the sensitivities to the biological reaction as indexes are used.

In step S16, the sensor control unit 67 controls on/off of sensing of the modals according to the reliabilities of the modals. For example, the sensor control unit 67 receives information regarding a sensor of a modal that does not contribute to user state estimation in the signal estimation, integration, and the like, supplied from the sensor signal processing unit 62 and the integration estimation unit 66, and notifies the sensor signal acquisition unit 61 of on/off of sensing corresponding to the modal according to the reliability of the modal.

After step S16, the user state estimation processing ends.

FIG. 5 is a flowchart illustrating the biological reaction sensitivity estimation processing in step S14 in FIG. 4.

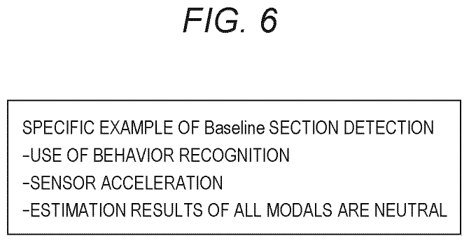

In step S31, the biological reaction sensitivity estimation unit 64 detects baseline sections of all target modals on the basis of the user state estimation result (estimated user state). Note that, as a specific example of the baseline section detection, the result of the behavior recognition, the acceleration that is information associated with the living body, a state in which the output results of all modals are neutral, and the like are used as illustrated in FIG. 6.

In step S32, the biological reaction sensitivity estimation unit 64 determines whether or not the baseline sections have been detected. In a case where it is determined in step S32 that the baseline sections of all modals have not been detected, yet, the processing returns to step S31, and the processing in step S31 and subsequent steps is repeated.

In a case where it is determined in step S32 that the baseline sections of all modals have been detected, the processing proceeds to step S33.

In step S33, the biological reaction sensitivity estimation unit 64 calculates a variation amount of the user state of each modal with the state in the baseline section as a base. Note that, when the variation amount of the user state from the state in the baseline section is calculated, a value obtained by multiplying the output (user state) of each modal by a preset correction coefficient α is used for sensitivity correction based on a type of application and physiological knowledge.

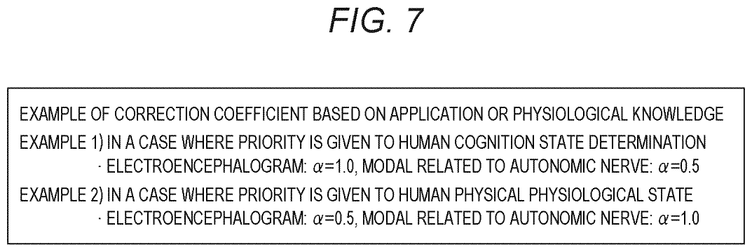

FIG. 7 is a diagram illustrating an example of the correction coefficient α based on a type of application and physiological knowledge.

As illustrated in FIG. 7, as an example 1, in a case where priority is given to human cognition determination, the correction coefficient α for the electroencephalogram is set to 1.0, and the correction coefficient α for the modals related to the autonomic nerve (for example, perspiration or heartbeat) is set to 0.5.

Furthermore, as an example 2, in a case where priority is given to human physical physiological state, the correction coefficient α for the electroencephalogram is set to 0.5, and the correction coefficient α for the modals related to the autonomic nerve is set to 1.0.

Returning to FIG. 5, in step S34, the biological reaction sensitivity estimation unit 64 determines whether or not the variation amount of the user state of a certain modal exceeds a threshold th1. In a case where it is determined in step S34 that the variation amount of the user state of a certain modal has not exceeded the threshold th1, the processing returns to step S33, and the processing in step S33 and subsequent steps is repeated.

In a case where it is determined in step S34 that the variation amount of the user state of a certain modal has exceeded the threshold th1, the processing proceeds to step S35.

In step S35, for other modals, which have the signal qualities equal to or more than a threshold th2, the biological reaction sensitivity estimation unit 64 calculates the variation amounts of the user states from the states in the baseline sections within fixed periods of the reaction time constants of the modals, and clusters the variation amounts of the user states of the target modals.

In step S36, the biological reaction sensitivity estimation unit 64 determines whether or not there exists a pair of cluster of one modal and a cluster of other modals, a distance between these clusters is being equal to or more than a threshold th3. At this time, the other modals are a plurality of modals. As long as the proportion of one modal to the other modals represents a small number of modals to a large amount of modals, one modal may not necessarily be 1, and may be 2.

In step S36, in a case where it is determined that there is not any pair of a cluster of one modal and a cluster of other modals, or even if there is a such pair of clusters, the distance between the clusters is not equal to or more than the threshold th3, the processing returns to step S31, and the processing in step S31 and subsequent steps is repeated.

In step S36, in a case where it is determined that there exists a cluster of one modal versus a cluster of other modals and the distance between these clusters is equal to or more than the threshold th3, the processing proceeds to step S37.

In step S37, the biological reaction sensitivity estimation unit 64 determines one modal as a modal having variation heterogeneity and the other modals as modals having no variation heterogeneity, and registers, in the biological reaction sensitivity DB 65, the information indicating the sensitivity to the biological reaction of each modal based on the determined variation heterogeneity. Thereafter, the processing returns to step S31, and the processing in step S31 and subsequent steps is repeated.

### 3. Second Embodiment (Early Fusion Type)

### <Second Configuration Example of User State Estimation Unit>

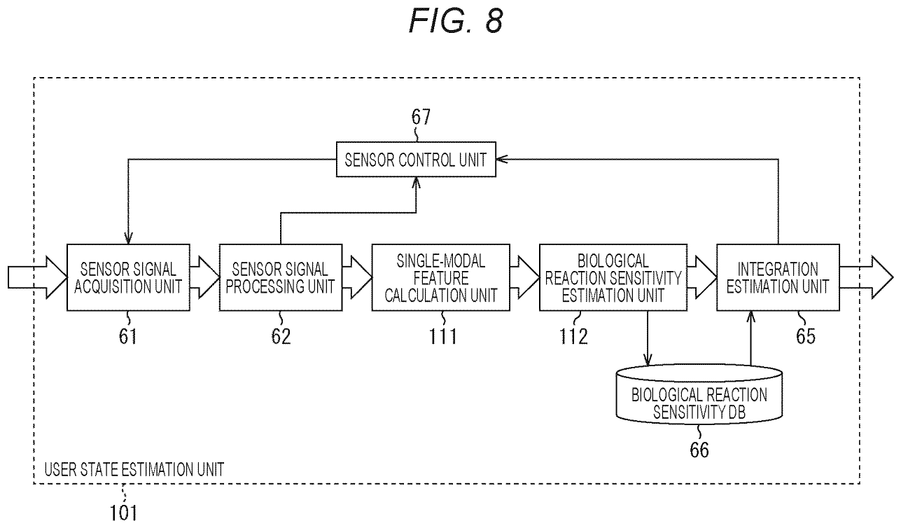

FIG. 8 is a block diagram illustrating a second configuration example of the user state estimation unit.

A user state estimation unit 101 of FIG. 8 is configured as an early fusion type that outputs the final user state estimation result by integrating the feature amount calculation results of the respective modals. Similarly to the user state estimation unit 51, the user state estimation unit 101 may be included in the biological information processing device 11, or may be included in the server 12 or the terminal device 13.

In FIG. 8, the user state estimation unit 101 includes the sensor signal acquisition unit 61, the sensor signal processing unit 62, a single-modal feature calculation unit 111, a biological reaction sensitivity estimation unit 112, the biological reaction sensitivity DB 65, the integration estimation unit 66, and the sensor control unit 67. Note that, portions common to those in FIG. 2 are denoted by corresponding reference signs.

The sensor signal processing unit 62 receives the biological signals from the sensor signal acquisition unit 61, performs preprocessing and signal quality estimation on the biological signals, and outputs, to the single-modal feature calculation unit 111, a set of the preprocessed signals and the information indicating the signal qualities that can be used in the subsequent processing.

The single-modal feature calculation unit 111 receives the preprocessed time-series signal and the information indicating the signal qualities supplied from the sensor signal processing unit 62, and calculates the feature amounts of the respective modals. The representative feature amount is the intensity of the a wave or the like for the electroencephalogram, and is heart rate variability (HRV) or the like for the pulse wave.

The single-modal feature calculation unit 111 outputs the calculated feature amounts of the respective modals to the biological reaction sensitivity estimation unit 112.

On the basis of the calculation result of the feature amounts of the single-modals supplied from the single-modal feature calculation unit 111, the biological reaction sensitivity estimation unit 112 detects a modal having variation heterogeneity at the feature amount level from modals. That is, the biological reaction sensitivity estimation unit 112 detects the baseline sections of all modals on the basis of the calculation result of the feature amounts of single-modals supplied from the single-modal feature calculation unit 111, detects variation amounts of the feature amounts with the feature amounts in the baseline sections as a base, and detects a modal having variation heterogeneity at the feature amount level of each modal on the basis of the detected variation amounts of the feature amounts. Then, the biological reaction sensitivity estimation unit 112 registers, in the biological reaction sensitivity DB 65, the information indicating the sensitivity to the biological reaction based on the presence or absence of the detected variation heterogeneity.

Note that, in the integration estimation unit 66 of FIG. 8, when outputting the emotion estimation result using the feature amounts of all modals, the contribution degree of the feature amounts with a low sensitivity is lowered on the basis of the sensitivity to the biological reaction of each modal indicated by the information registered in the biological reaction sensitivity DB 65. That is, the integration estimation unit 66 adjusts the contribution degrees of the feature amounts for the respective modals on the basis of the reliabilities of the feature amounts for the respective modals using the signal qualities and the sensitivities to the biological reaction as indexes, thereby integrally estimating the state of the user. As a result, the user state is estimated with high accuracy.

### <Processing of User State Estimation Unit>

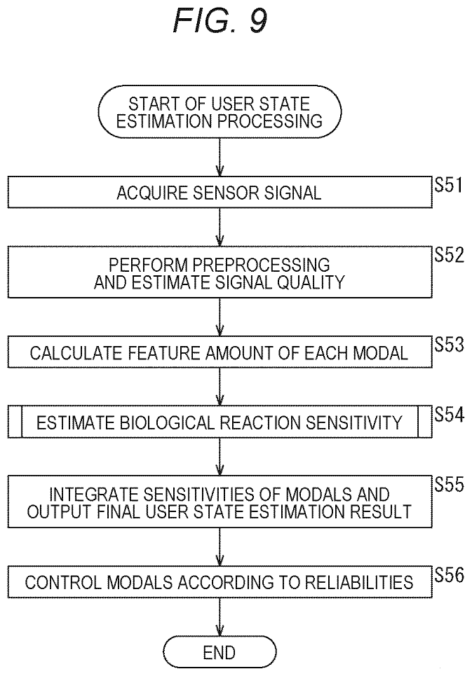

FIG. 9 is a flowchart illustrating user state estimation processing of the user state estimation unit 101 of FIG. 8.

Note that, the processing in steps S51, S52, S55, and S56 in FIG. 9 is basically similar to the processing in steps S11, S12, S15, and S16 in FIG. 4, and thus the descriptions thereof will be omitted to avoid the repetition.

In step S53, the single-modal feature calculation unit 111 receives the processed time-series signal and the information indicating the signal quality supplied from the sensor signal processing unit 62, and calculates the feature amount for each modal.

The single-modal feature calculation unit 111 outputs, together with the feature amount for each modal, the information indicating the quality of the input signal used for calculation to the subsequent biological reaction sensitivity estimation unit 112.

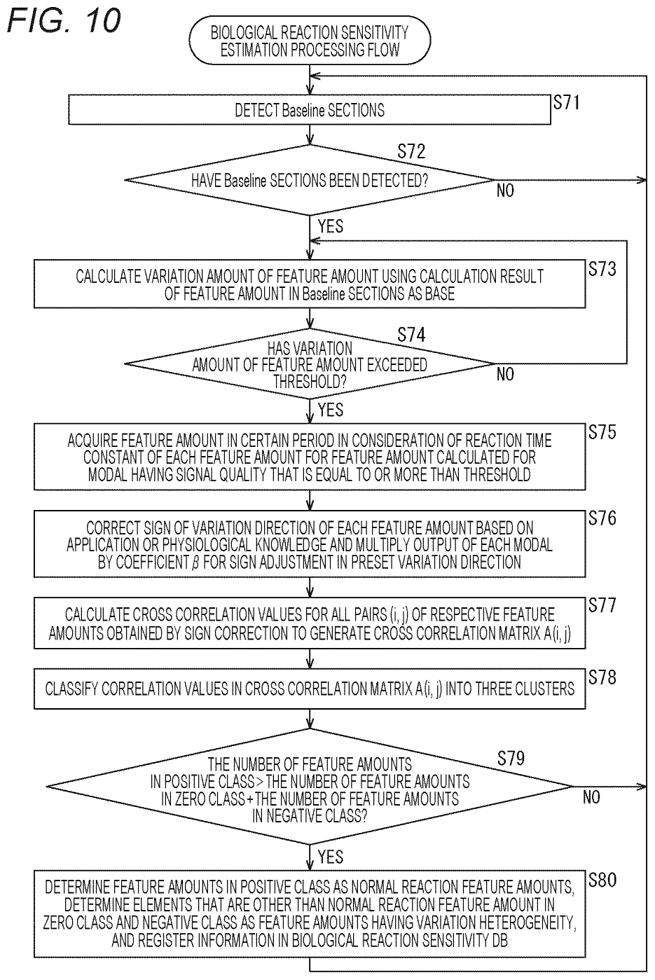

In step S54, the biological reaction sensitivity estimation unit 112 performs biological reaction sensitivity estimation processing. Details of the biological reaction sensitivity estimation processing will be described later with reference to FIG. 10. By the processing of step S54, the baseline sections of all modals are detected, variation amounts of feature amounts are detected with the feature amounts in the baseline sections as a base, a modal having variation heterogeneity from a plurality of the modals is detected on the basis of the variation amounts of the feature amounts, and the information indicating the sensitivity to the biological reaction of each modal based on the presence or absence of the variation heterogeneity is registered in the biological reaction sensitivity DB 65.

In step S55, the integration estimation unit 66 integrates the feature amounts for the respective modals supplied from the single-modal feature calculation unit 111 and the sensitivities of the respective modals supplied from the biological reaction sensitivity estimation unit 112 or indicated by the information registered in the biological reaction sensitivity DB 65, and outputs the final user state estimation result. At this time, for the integration, the reliabilities of the feature amounts for the respective modals using the signal qualities and the sensitivities to the biological reaction as indexes are used.

FIG. 10 is a flowchart illustrating the biological reaction sensitivity estimation processing in step S54 in FIG. 9.

In step S71, the biological reaction sensitivity estimation unit 112 detects baseline sections of all target modals on the basis of the calculation result of the feature amounts for single-modals supplied from the single-modal feature calculation unit 111.

In step S72, the biological reaction sensitivity estimation unit 112 determines whether or not the baseline sections have been detected. In a case where it is determined in step S32 that the baseline sections of all modals have not been detected yet, the processing returns to step S71, and the processing in step S71 and subsequent steps is repeated.

In a case where it is determined in step S72 that the baseline sections of all modals have been detected, the processing proceeds to step S73.

In step S73, the biological reaction sensitivity estimation unit 112 calculates a variation amount of a feature amount using the feature amount in the baseline sections as a base.

In step S74, the biological reaction sensitivity estimation unit 112 determines whether or not the variation amount of the feature amount exceeds a threshold th11. In a case where it is determined in step S74 that the variation amount of the feature amount does not exceed the threshold th11, the processing proceeds to step S75.

In step S75, the biological reaction sensitivity estimation unit 112 acquires a feature amount in a fixed period in consideration of a reaction time constant of each feature amount for the feature amount calculated for a modal having the signal quality that is equal to or more than the threshold th11.

In step S76, the biological reaction sensitivity estimation unit 112 corrects a sign of a variation direction of each feature amount based on a type of application and physiological knowledge.

For example, the biological reaction sensitivity estimation unit 112 multiplies the output (feature amount) of each modal by a coefficient β for sign correction of a preset variation direction. For example, in an application that estimates the wakefulness level of the user, in a case where the wakefulness degree is increased, an increasing feature amount is multiplied by a coefficient β=1, and a decreasing feature amount is multiplied by a coefficient β=−11.

In step S77, the biological reaction sensitivity estimation unit 112 calculates cross correlation values for all pairs (i, j) of the respective feature amounts obtained by the sign correction to generates a cross correlation matrix A(i, j).

In step S78, the biological reaction sensitivity estimation unit 112 classifies the correlation values in the generated cross correlation matrix A(i, j) into three clusters. For example, in a case where the correlation values are classified into clusters of a positive class, a zero class, and a negative class, for example, TH1=0.2, TH2=−0.2, and the like are used as classification thresholds.

In step S79, the biological reaction sensitivity estimation unit 112 determines whether or not the number of feature amounts of the positive class is more than the number of feature amounts of the zero class+the number of feature amounts of the negative class. The number of feature amounts are weighted for uniformity between the modals and counted.

In a case where it is determined in step S79 that the number of feature amounts of the positive class is equal to or less than the number of feature amounts of the zero class+the number of feature amounts of the negative class, the processing returns to step S71, and the processing in step S71 and subsequent steps is repeated.

In a case where it is determined in step S79 that the number of feature amounts of the positive class is equal to or more than the number of feature amounts of the zero class+the number of feature amounts of the negative class, the processing proceeds to step S80.

In step S80, the biological reaction sensitivity estimation unit 112 determines elements (feature amounts) in the positive class as a normal reaction feature amount, and determines elements that are other than the normal reaction feature amounts in the zero class and the negative class as feature amounts having variation heterogeneity. The biological reaction sensitivity estimation unit 112 registers, in the biological reaction sensitivity DB 65, the information indicating the sensitivities to the biological reaction of the respective modals based on modals having the normal reaction feature amounts and the feature amounts having variation heterogeneity that are determined.

After step S80, the processing returns to step S71, and the processing in step S71 and subsequent steps is repeated.

Note that, in the above description, examples of the late fusion type user state estimation unit and the early fusion type user state estimation unit have been described, but the user state estimation unit may be configured by combining the late fusion type and the early fusion type. In this case, the direction of the sensitivity estimation may be calculated at the feature amount level in the early fusion type, and the calculated direction may be corrected and the sensitivity estimation may be integrated in the late fusion type.

### 4. Use Case

### <Application Using Estimation Result of User State>

Examples of the application that uses the estimation result of the user state described above include the follows.
Detection and treatment of diseases and disabilities, and support therefor
Education (planning the degree of understanding and concentration)
Game, movie, and entertainment (user's reaction is referenced in content production)
Advertisement and retail (targeting)
Recruitment or hiring (comfort or discomfort feeling detection in an interview)
Call center voice assistant
Stress detection, stress release, and stress coping
Automobile and industrial safety (detection of fatigue and doze during driving and work)
Detection of wonder and intervention in law enforcement (suicide detection and immigration inspection)
Community, politics, and social network
Social robot (emotional connection)

### <Specific Use Case>

Next, as a use case, a learning efficiency improvement support system in a case where the user state estimation unit 51 of FIG. 2 is used in an application for learning efficiency improvement support will be described.

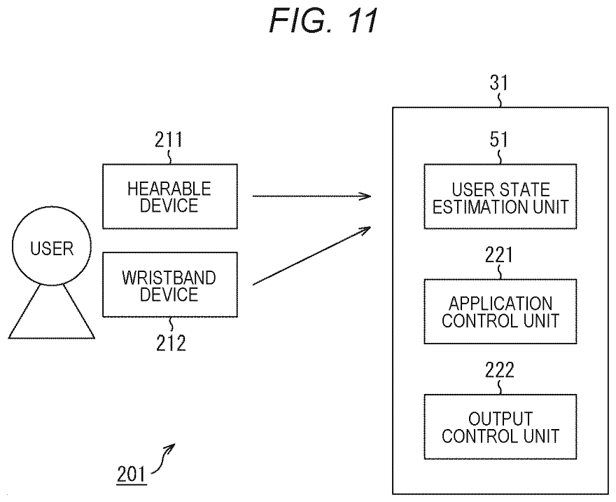

FIG. 11 is a diagram illustrating a learning efficiency improvement support system to which the present technology is applied.

A learning efficiency improvement support system 201 of FIG. 11 includes a hearable device 211, a wristband device 212, and the terminal device 13 of FIG. 1.

In the learning efficiency improvement support system 201, the hearable device 211 and the wristband device 212 are connected to the terminal device 13 by wireless communication. Furthermore, at the time of learning start, the hearable device 211 is worn on both ears of a user, and the wristband device 212 is worn on a wrist of the user.

The hearable device 211 is an earphone-type device worn on both ears, can acquire an ear portion EEG signal (hereinafter referred to as a H-E) and a PPG signal (hereinafter referred to as a H-P) as modal biological signals, and can acquire an acceleration (ACC) (hereinafter, referred to as a H-A) as information associated with the living body.

The wristband device 212 is a smart watch worn on the wrist, can acquire a wrist EDA signal (hereinafter referred to as a W-E) and a PPG signal (hereinafter, referred to as a W-P) as modal biological signals, and can acquire an acceleration (ACC) (hereinafter, referred to as a W-A) as information associated with the living body.

The terminal device 13 includes the user state estimation unit 51 of FIG. 2, an application control unit 221, and an output control unit 222 by activation of an application for learning efficiency improvement support.

The H-E, H-P, and H-A acquired by the hearable device 211 are transmitted to the terminal device 13. The W-E, W-P, and W-A acquired by the wristband device 212 are transmitted to the terminal device 13.

After the learning start, the user state estimation unit 51 estimates the user state on the basis of the acquired modal biological signals and the information associated with the living body.

An application control unit 121 controls notification to the user according to the user state. The notification to the user is, for example, a phone call, an email, a message, and a notification to the user from the application itself and other applications, a system, or the like.

In a case where the degree of concentration of the user is high, the application control unit 121 suspends the notification other than the notification with high urgency and importance. On the other hand, in a state where the degree of concentration of the user is low, the application control unit 121 controls an output control unit 122 to perform notification with high importance regardless of the level of urgency.

Furthermore, in a case where a state in which the degree of concentration of the user is high continues for a fixed period and a state in which the stress of the user is high is detected, the application control unit 121 controls the output control unit 122 to notify the user of a proposal for a break.

The output control unit 122 controls an output unit including an LCD, a speaker, and the like under the control of the application control unit 121.

### <Details of Use Case>

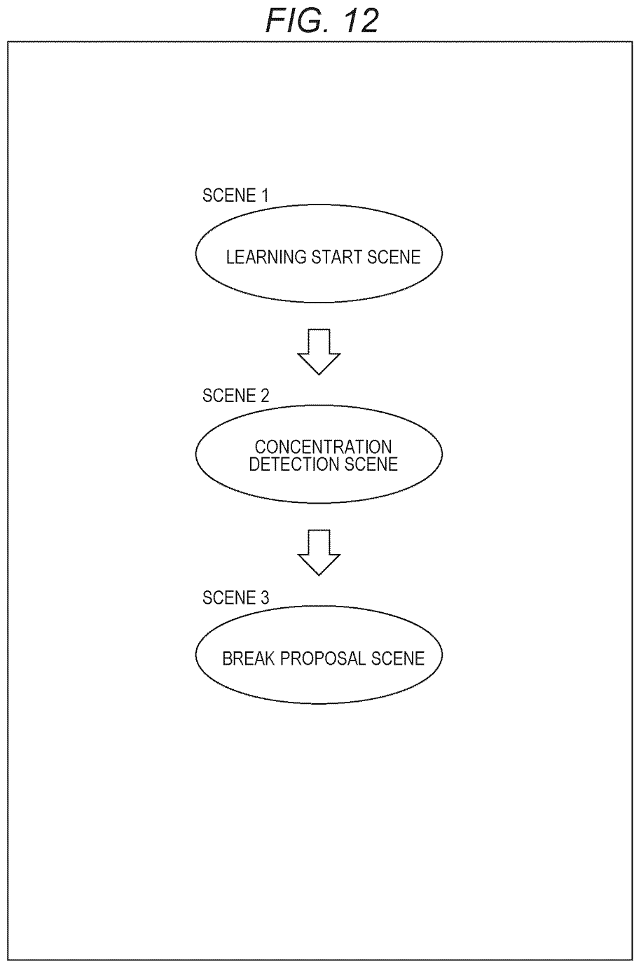

FIG. 12 is a diagram illustrating each scene in a use case. In the use case, as illustrated in FIG. 12, the scene can be divided into three scenes of a learning start scene which is scene 1, a concentration detection scene which is scene 2, and a break proposal scene which is scene 3.

### <Processing in Learning Start Scene>

First, processing in scene 1, which is the learning start scene will be described.

In the learning start scene, for example, the user starts an application for learning efficiency improvement support on the terminal device 13 and starts learning in a state where both the hearable device 211 and the wristband device 212 are normally worn.

The sensor signal acquisition unit 61 of the user state estimation unit 51 acquires the H-E, H-P, and H-A transmitted from the hearable device 211, and acquires the W-E, W-P, and W-A transmitted from the wristband device 212. The H-E, H-P, H-A, W-E, W-P, and W-A are supplied to the sensor signal processing unit 62.

The sensor signal processing unit 62 performs preprocessing and signal quality estimation on the biological signals of modals (H-E, H-P, W-E, and W-P) received from the sensor signal acquisition unit 61 to confirm that the signal quality of the biological signals of all modals is good. The sensor signal processing unit 62 outputs a set of the preprocessed signals and the signal qualities to the single-modal emotion estimation unit 63.

The single-modal emotion estimation unit 63 receives the preprocessed time-series signal and the signal qualities supplied from the sensor signal processing unit 62, and performs user state estimation for single-modals using estimation models corresponding to the modals. Note that, since it is immediately after the start, it is estimated that all modals are neutral.

Together with the user state estimation results, the single-modal emotion estimation unit 83 outputs the qualities of the input signals used for the estimation to the biological reaction sensitivity estimation unit 64.

The biological reaction sensitivity estimation unit 64 starts detection of baseline sections of all modals, detects a modal having variation heterogeneity of the user state from a plurality of modals on the basis of the variation properties and the variation degrees from the states in the baseline sections in consideration of the reaction time constants for the respective modals, and registers, in the biological reaction sensitivity DB 65, the information indicating the sensitivity to the biological reaction for each modal based on the presence or absence of the variation heterogeneity.

The integration estimation unit 66 integrates the single-modal emotion estimation results supplied from the single-modal emotion estimation unit 63 and the sensitivities of the respective modals stored in the biological reaction sensitivity DB 65, lowers the reliability of the W-E because the sensitivity of the W-E is poor, and then outputs the integrated estimation result to the application control unit 121.

### <Processing in Concentration Detection Scene>

Next, processing in scene 2, which is the concentration detection scene of will be described.

The scene transitions from the learning start scene described above to the concentration detection scene. In the concentration detection scene, the user starts to concentrate on study.

As a result of user state estimation of the H-E for a single-modal by the single-modal emotion estimation unit 63, the concentration state of the user is estimated. The biological reaction sensitivity estimation unit 64 determines that the H-E has exceeded a threshold from the state in the baseline section, buffers the change in the estimation states of the respective modals for a time length enough for the PPG signals (H-P and W-P) having the longest reaction time constant for the other modals, and determines the presence or absence of variation heterogeneity from the modals on the basis of the variation properties and the variation degrees.

At that time, it is assumed that, both PPG signals (H-P and W-P) vary from the state in the baseline section toward the concentration similarly to H-E, but the W-E does not vary.

Then, the biological reaction sensitivity estimation unit 64 registers, in the biological reaction sensitivity DB 65, the information indicating the sensitivities to the biological reaction of the respective modals based on the variation heterogeneity. The integration estimation unit 66 integrates the estimation results of the user states of the single-modals supplied from the single-modal emotion estimation unit 63 and the sensitivities of the respective modals indicated by the information registered in the biological reaction sensitivity DB 65, lowers the reliability of the W-E, and outputs, to the application control unit 121, the integrated estimation result obtained by integrating the estimation results of the other three modals.

In the current integration, since the reliability of the W-E is low, the integration estimation unit 66 notifies the sensor control unit 67 of temporary turn-off of the sensing of the W-E.

### <Processing in Break Proposal Scene>

Finally, processing in scene 3, which is the break proposal scene will be described.

The scene transitions from the concentration detection scene described above to the break proposal scene.

In the break proposal scene, it is detected that the high concentration state of the user continues for a long time and the stress of the user is high.

When the high stress state of the user continues for a fixed period of time, the biological reaction sensitivity estimation unit 64 detects the high stress state of the user as the baseline section.

For example, the fact that the high stress state of the user continues is output to the application control unit 121 via the integration estimation unit 66. The application control unit 121 controls the output control unit 122 such that a break proposal for the user is displayed on a display (not illustrated).

When the user who sees the proposal accepts the break and stretches, the wearing state of the wristband device 212 on the wrist changes, and the signal quality of the W-P deteriorates.

The biological reaction sensitivity estimation unit 64 detects the variation to a resting state from a high stress state in the baseline section of the H-E, and buffers the variations of the other modals for a fixed period.

Since the W-E is off and the signal quality of the W-P continues to be low, the biological reaction sensitivity estimation unit 64 checks the variation of the H-P to confirm that the two modals have similar variations, and registers, in the biological reaction sensitivity DB 65, the information indicating the sensitivities to the biological reaction based on the absence of variation heterogeneity.

The integration estimation unit 66 integrates the emotion estimation results of the single-modals supplied from the single-modal emotion estimation unit 63 and the sensitivities of the respective modals indicated by the information registered in the biological reaction sensitivity DB 65, and outputs, to the application control unit 121, the integration estimation result obtained by integrating the estimation results of the two modals.

If the estimation results of the two modals do not match, neither of them can be said to have variation heterogeneity, thus the integration estimation unit 66 outputs the integration estimation result obtained by integrating the estimation results.

Thereafter, the scene returns to scene 1, which is the learning start scene, and the subsequent processing is repeated until the user instructs termination.

As described above, according to the learning efficiency improvement support system 201, since the user can alternately perform concentrated learning and break, the user can learn efficiently.

As described above, in the present technology, on the basis of an application or physiological knowledge, a modal having variation heterogeneity from the modals is estimated on the basis of at least one of the variation properties or the variation amounts of the estimation results of the user states of the respective modals from the baseline sections.

That is, according to the present technology, since the individual characteristics of variation heterogeneity is considered, so that it is possible to enhance the integration accuracy of the user state estimation using multimodal.

Furthermore, in the present technology, the estimation result of a modal having variation heterogeneity is set as the sensitivity to the physiological reaction for each individual, and is incorporated into the reliability of the estimation result together with the signal quality of the modal.

As a result, the result of the user state estimation obtained by integrating the modals can be personalized, enabling contribution to improvement in estimation accuracy.

Furthermore, according to the present technology, a modal that does not contribute to estimation of the user state at the time of integration is detected, and sensing is dynamically turned off.

As a result, in a system requiring power saving such as a wearable environment, power saving can be realized while the estimation accuracy of the user state is maintained.

### 5. Others

### <Effects of Present Technology>

In the present technology, the signal quality is estimated for each modal representing a type of the biological signal of a user, a modal having variation heterogeneity representing that variation of the biological signal is different from a plurality of modals is detected, the sensitivity to the biological reaction of the modal based on the detection result of the modal having the variation heterogeneity is estimated, and the state of the user is integrally estimated on the basis of the signal qualities and the sensitivities to the biological reaction.

This can improve the estimation accuracy of the user state.

### <Configuration Example of Computer>

The series of processing steps described above can be executed by hardware and also can be executed by software. In a case where the series of processing steps is executed by software, a program included in the software is installed from a program recording medium on a computer incorporated in dedicated hardware, a general-purpose personal computer, or the like.

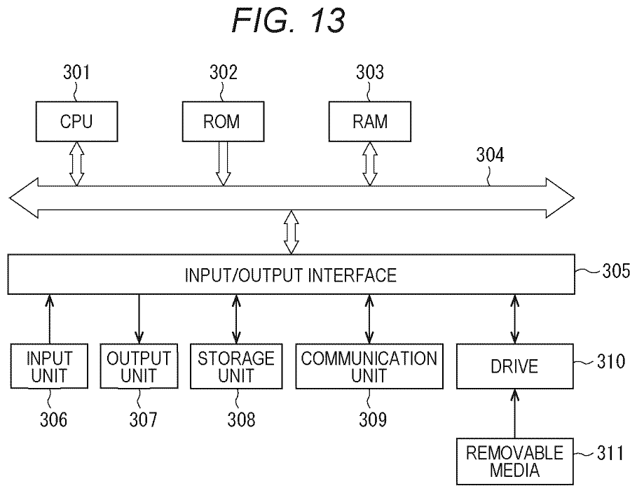

FIG. 13 is a block diagram illustrating a configuration example of hardware of a computer that executes the above-described series of processing steps by a program.

A central processing unit (CPU) 301, a read only memory (ROM) 302, and a random access memory (RAM) 303 are connected to each other by a bus 304.

Furthermore, to the bus 304, an input/output interface 305 is connected. To the input/output interface 305, an input unit 306 including a keyboard, a mouse, and the like, and an output unit 307 including a display, a speaker, and the like are connected. In addition, to the input/output interface 305, a storage unit 308 including a hard disk, a nonvolatile memory, and the like, a communication unit 309 including a network interface and the like, and a drive 310 that drives a removable medium 311 are connected.

In the computer configured as described above, the above-described series of processing steps is executed, for example, by the CPU 301 loading the program stored in the storage unit 308 into the RAM 303 via the input/output interface 305 and the bus 304 and executing the program.

The program to be executed by the CPU 301 is provided, for example, by being recorded on the removable medium 311 or via a wired or wireless transmission medium such as a local area network, the Internet, or digital broadcasting, and is installed on the storage unit 308.

Note that, the program to be executed by the computer may be a program causing processing steps to be performed in time series in the order described in the present specification, or may be a program causing processing steps to be performed in parallel or at necessary timing such as when a call is made, or the like.

Note that, in the present specification, a system means an assembly of a plurality of components (such as devices and modules (parts)) and it does not matter whether or not all the components are in the same housing. Therefore, a plurality of devices housed in separate housings and connected to each other via a network and one device in which a plurality of modules is housed in one housing are both systems.

In addition, the effects described in the present specification are merely examples and not restrictive, and there may also be other effects.

An embodiment of the present technology is not limited to the embodiment described above, and various modifications can be made without departing from the scope of the present technology.

For example, the present technology may be configured as cloud computing in which one function is shared by a plurality of devices via a network and processed in cooperation.

Furthermore, each step described in the above flowcharts can be performed by one device, or can be performed in a shared manner by a plurality of devices.

Furthermore, in a case where a plurality of processing steps is included in one step, the plurality of processing steps included in the one step can be performed by one device or can be performed in a shared manner by a plurality of devices.

### <Combination Example of Configuration>

The present technology can have the following configurations.

(1)

A signal processing apparatus including:
a signal processing unit that estimates signal qualities for respective modals each representing a type of a biological signal of a user;
a sensitivity estimation unit that detects at least one of the modals having variation heterogeneity representing that variation of the biological signal is different from a plurality of the modals, and estimates a sensitivity to biological reaction of the modal based on a detection result of the modal having the variation heterogeneity; and
an integration estimation unit that integrally estimates a state of the user on the basis of the signal qualities and the sensitivities to the biological reaction.
   (2)

The signal processing apparatus according to (1), further including:
a single-modal state estimation unit that estimates states of the user for the respective modals, in which
the sensitivity estimation unit detects the modal having the variation heterogeneity on the basis of variation amounts of the states of the user for the respective modals, and
the integration estimation unit integrally estimates the state of the user by integrating the states of the user for the respective modals on the basis of the signal qualities and the sensitivities to the biological reaction.
   (3)

The signal processing apparatus according to (2), in which
the sensitivity estimation unit detects a baseline section indicating a section where a state of the user is stable from among states of the user for each modal, and detects the modal having the variation heterogeneity on the basis of the variation amount of the state of the user for each modal calculated with the state of the user in the baseline section as a base.
   (4) The signal processing apparatus according to (3), in which
when calculating the variation amount of the state of the user for each modal, on the basis of a type of application or physiological knowledge, the sensitivity estimation unit uses a value obtained by multiplying a reliability of the state of the user for the corresponding modal by a preset correction coefficient.
   (5)

The signal processing apparatus according to (2), in which
the integration estimation unit integrally estimates the state of the user by integrating the states of the user for the respective modals on the basis of reliabilities of the states of the user for the respective modals using the signal qualities and the sensitivities to the biological reaction as indexes.
   (6)

The signal processing apparatus according (5), further including:
a sensor control unit that controls stop of sensing of the biological signal of at least one of the modals for which the reliability of the state of the user for the corresponding modal is estimated to be lower than a threshold.
   (7)

The signal processing apparatus according to (1), further including:
a single-modal feature amount calculation unit that calculates feature amounts of the biological signals of the respective modals, in which, the sensitivity estimation unit detects the modal having the variation heterogeneity on the basis of variation amounts of the feature amounts for the respective modals, and
the integration estimation unit integrally estimates the state of the user using the feature amounts for the respective modals on the basis of the signal qualities and the sensitivities to the biological reaction.
   (8)

The signal processing apparatus according to (7), in which
the sensitivity estimation unit detects a baseline section indicating a section where a feature amount of the user is in a stable state from among feature amounts of the user for each modal, and detects the modal having the variation heterogeneity on the basis of the variation amount of the feature amount of the user for each modal calculated with the feature amount of the user in the baseline section as a base.
   (9)

The signal processing apparatus according to (8), in which
when calculating the variation amount of the feature amount for each modal, on the basis of a type of application or physiological knowledge, the sensitivity estimation unit uses a value obtained by multiplying the feature amount for the corresponding modal by a coefficient of sign correction of a preset variation direction.
   (10)

The signal processing apparatus according to (7), in which
the integration estimation unit integrally estimates the state of the user by adjusting contribution degrees of the feature amounts for the respective modals on the basis of reliabilities of the feature amounts for the respective modals using the signal qualities and the sensitivities to the biological reaction as indexes.
   (11)

The signal processing apparatus according to (10), further including:
a sensor control unit that controls stop of sensing of the biological signal of at least one of the modals for which the reliability of the state of the user for the corresponding modal is estimated to be lower than a threshold.
   (12)

The signal processing apparatus according to any one of (1) to (11), in which
the sensitivity estimation unit registers information indicating estimated sensitivities to the biological reaction of the modals in a database, and
the integration estimation unit integrally estimates the state of the user on the basis of the signal qualities and the sensitivities to the biological reaction indicated by information registered in the database.
   (13)

The signal processing apparatus according to any one of (1) to (12), in which
the variation heterogeneity represents that at least one of a property or a degree of the variation of the biological signal is different.
   (14)

The signal processing apparatus according to (1), further including:
a sensor control unit that controls stop of sensing of the biological signal of at least one of the modals for which the signal quality is estimated to be worse than a threshold.
   (15)

A signal processing method performed by a signal processing apparatus, including:
estimating signal qualities for respective modals each representing a type of a biological signal of a user;
detecting at least one of the modals having variation heterogeneity representing that variation of the biological signal is different from a plurality of the modals, and estimating a sensitivity to biological reaction of the modal based on a detection result of the modal having the variation heterogeneity; and
integrally estimating a state of the user on the basis of the signal qualities and the sensitivities to the biological reaction.

### REFERENCE SIGNS LIST

1 Emotion estimation processing system
11 Biological information processing device
12 Server
13 Terminal device
14 Network
51 User state estimation unit
61 Sensor signal acquisition unit
62 Sensor signal processing unit
63 Single-modal emotion estimation unit
64 Biological reaction sensitivity estimation unit
65 Biological reaction sensitivity DB
66 Integration estimation unit
67 Sensor control unit
81 Preprocessing unit
82 Signal quality estimation unit
101 User state estimation unit
111 Single-modal feature calculation unit
112 Biological reaction sensitivity estimation unit
201 Learning efficiency improvement support system
211 Hearable device
212 Wristband device
221 Application control unit
222 Output control unit

1. A signal processing apparatus comprising:
a signal processing unit that estimates signal qualities for respective modals each representing a type of a biological signal of a user;
a sensitivity estimation unit that detects at least one of the modals having variation heterogeneity representing that variation of the biological signal is different from a plurality of the modals, and estimates a sensitivity to biological reaction of the modal based on a detection result of the modal having the variation heterogeneity; and
an integration estimation unit that integrally estimates a state of the user on a basis of the signal qualities and the sensitivities to the biological reaction.

2. The signal processing apparatus according to claim 1, further comprising:
a single-modal state estimation unit that estimates states of the user for the respective modals, wherein
the sensitivity estimation unit detects the modal having the variation heterogeneity on a basis of variation amounts of the states of the user for the respective modals, and
the integration estimation unit integrally estimates the state of the user by integrating the states of the user for the respective modals on a basis of the signal qualities and the sensitivities to the biological reaction.

3. The signal processing apparatus according to claim 2, wherein
the sensitivity estimation unit detects a baseline section indicating a section where a state of the user is stable from among states of the user for each modal, and detects the modal having the variation heterogeneity on a basis of the variation amount of the state of the user for each modal calculated with the state of the user in the baseline section as a base.

4. The signal processing apparatus according to claim 3, wherein
when calculating the variation amount of the state of the user for each modal, on a basis of a type of application or physiological knowledge, the sensitivity estimation unit uses a value obtained by multiplying a reliability of the state of the user for the corresponding modal by a preset correction coefficient.

5. The signal processing apparatus according to claim 2, wherein
the integration estimation unit integrally estimates the state of the user by integrating the states of the user for the respective modals on a basis of reliabilities of the states of the user for the respective modals using the signal qualities and the sensitivities to the biological reaction as indexes.

6. The signal processing apparatus according to claim 5, further comprising:
a sensor control unit that controls stop of sensing of the biological signal of at least one of the modals for which the reliability of the state of the user for the corresponding modal is estimated to be lower than a threshold.

7. The signal processing apparatus according to claim 1, further comprising:
a single-modal feature amount calculation unit that calculates feature amounts of the biological signals of the respective modals, wherein the sensitivity estimation unit detects the modal having the variation heterogeneity on a basis of variation amounts of the feature amounts for the respective modals, and
the integration estimation unit integrally estimates the state of the user using the feature amounts for the respective modals on a basis of the signal qualities and the sensitivities to the biological reaction.

8. The signal processing apparatus according to claim 7, wherein
the sensitivity estimation unit detects a baseline section indicating a section where a feature amount of the user is in a stable state from among feature amounts of the user for each modal, and detects the modal having the variation heterogeneity on a basis of the variation amount of the feature amount of the user for each modal calculated with the feature amount of the user in the baseline section as a base.

9. The signal processing apparatus according to claim 8, wherein
when calculating the variation amount of the feature amount for each modal, on a basis of a type of application or physiological knowledge, the sensitivity estimation unit uses a value obtained by multiplying the feature amount for the corresponding modal by a coefficient of sign correction of a preset variation direction.

10. The signal processing apparatus according to claim 7, wherein
the integration estimation unit integrally estimates the state of the user by adjusting contribution degrees of the feature amounts for the respective modals on a basis of reliabilities of the feature amounts for the respective modals using the signal qualities and the sensitivities to the biological reaction as indexes.

11. The signal processing apparatus according to claim 10, further comprising:
a sensor control unit that controls stop of sensing of the biological signal of at least one of the modals for which the reliability of the state of the user for the corresponding modal is estimated to be lower than a threshold.

12. The signal processing apparatus according to claim 1, wherein
the sensitivity estimation unit registers information indicating estimated sensitivities to the biological reaction of the modals in a database, and
the integration estimation unit integrally estimates the state of the user on a basis of the signal qualities and the sensitivities to the biological reaction indicated by information registered in the database.

13. The signal processing apparatus according to claim 1, wherein
the variation heterogeneity represents that at least one of a property or a degree of the variation of the biological signal is different.

14. The signal processing apparatus according to claim 1, further comprising:
a sensor control unit that controls stop of sensing of the biological signal of at least one of the modals for which the signal quality is estimated to be worse than a threshold.

15. A signal processing method performed by a signal processing apparatus, comprising:
estimating signal qualities for respective modals each representing a type of a biological signal of a user;
detecting at least one of the modals having variation heterogeneity representing that variation of the biological signal is different from a plurality of the modals, and estimating a sensitivity to biological reaction of the modal based on a detection result of the modal having the variation heterogeneity; and
integrally estimating a state of the user on a basis of the signal qualities and the sensitivities to the biological reaction.
